(19) United States
(12) Patent Application Publication
(10) Pub. No.: US 2003/0031723 A1
(43) Pub. Date: Feb. 13, 2003

(54) Formulation and method for preparation of rapid dispersion spheroids which can be heavily titrated

(76) Inventors: Iderne Michel, Strasbourg (FR); Olivier Jacob, Montpellier (FR); Bernard Bataille, Saint Gely Du Fesc (FR); Maurice Jacob, Montpellier (FR)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/201,871

(22) Filed: Jul. 23, 2002

(30) Foreign Application Priority Data
Jul. 25, 2001 (FR) 01 09912

(51) Int. Cl. A61K 9/16; A61K 9/50; A61K 35/78

(52) U.S. Cl. 424/494; 424/725

(57) ABSTRACT

The invention concerns the formulation and method of fabricating rapid dispersion spheroids which can be heavily charged with active substances. These spheroids are formulated using at least one active substance, specifically a solution of vegetable origin, absorbed and/or adsorbed on a dry technological mixture with a slightly substituted hydroxypropylated cellulose ether base at the level of core groups β-O-glucopyranosil, preferably characterized by having a substitution rate of about 11% and a water soluble fractional percentage of about 4.29%. The spheroids are obtained by an extrusion and spheronization process. This invention is of interest in the fields of health, hygiene, dietetics, cosmetology, nutrition, and agriculture; it may concern humans or animals.

The present invention concerns the formulation of slightly substituted hydroxypropylene cellulose ether-based spheroids that are capable of rapid dispersion and can be heavily charged with active substances.

It also concerns the method of preparing these slightly substituted hydroxypropylene cellulose ether-based, rapid dispersion spheroids which permits the addition of a highly concentrated quantity of an active solution, specifically a solution of vegetable origin, to a spheroid.

The invention further concerns compounds of vegetable, mineral, vitamin, optionally with an active organic component base, in the form of this type of spheroid.

Finally, it concerns derivative applications for the internal or external administration of these spheroids, particularly in the fields of health, hygiene, dietetics, cosmetology, nutrition, and agriculture, either human or animal.

One goal of the invention is to furnish spheroids that are heavily titrated with active principles.

Another goal of the present invention is to furnish rapid dispersion spheroids.

The preparation of active solutions in liquid form is well known in the art, particularly solutions with a vegetable product base obtained through extraction, maceration, infusion, decoction, digestion or lixiviation.

However, in direct use these liquid extracts demonstrate numerous disadvantages, particularly with respect to their physical and chemical instability during storage, their characteristically low content in vegetable constituents, and the frequent occurrence of a fairly high level of ethanol, which is generally undesirable for oral administration of medicinal products.

Furthermore, a liquid form is not very practical to use. Dosage and measurement of the product are complicated, it is difficult to transport, and it can be accidentally spilled.

For all of these reasons, a solid form is highly desirable.

Current methods of transforming these liquid extracts into dry extracts having fewer disadvantages utilize significant amounts heat such as, for example, nebulization, drying on rotating cylinders, or evaporation under reduced pressure.

While these methods produce dry components in powder form that are easier to use and in particular, can be administered orally, they require high temperatures which can alter the fragile active principles, such as, for example, the constituent characteristics of vegetable products.

Moreover, these dry extracts are often hygroscopic, causing absorption of humidity and clumping which can alter physical and chemical stability and cause problems in reproducibility and manipulation.

There is also the method described in French Patent No. F.R. 2.721.512 which consists of absorbing and adsorbing a solution of vegetable origin on a powder solution generally of a natural or synthetic polymer type, extruding, and then spheronizing the wetted mass in order to form spheroids.

With this method, simplified formulations are obtained advantageously without thermal degradation, allowing liquid preparations of vegetable origin to be administered in dry form and ensuring physical and chemical stability over time of the active principles used.

The polymers described in this prior process as possessing suitable absorbent and adsorbent properties are microcrystalline cellulose, micro fine cellulose, starches, modified starches and polysaccharides, with microcrystalline cellulose being preferred. These technological excipients possess satisfactory plastic qualities and suitable absorption and adsorption properties.

However, it is especially advantageous to improve these properties in order to obtain spheroids that are more highly concentrated in active principles.

It is actually possible to add more of the active principle to a smaller distribution system. Thus, for example, with an orally administered substance, it is possible to decrease the number of doses per day or the overall amount of the total material swallowed in each dose.

These objectives have been attained through the formulation and the method of the invention.

For this purpose, according to an essential feature of the invention, a specific technical excipient is used which, surprisingly, has been shown to be particularly advantageous as both an adsorption and an absorption substrate for the active principles and which is compatible with the extrusion spheronization method.

The method of making spheroids heavily titrated with active principles according to the invention comprises the following steps:

furnishing a highly concentrated solution or active powder;

furnishing a dry, homogeneous mixture of slightly substituted cellulosic hydroxypropylated polymer with very high absorbent and adsorbent properties;

wetting the dry mixture with the active solution or with another aqueous or non-aqueous liquid;

extrusion;

forming spheroids by spheronizing the extruded product;

drying; and

calibrating the dried compound to form multiparticular spheroid systems of controlled granular size.

The method of the invention may comprise a supplemental step consisting of:

coating these multiparticular systems with a film to protect their constituents or modify the extent to which they are released within the organism.

The method of the invention allows any active vegetable compound in liquid form, specifically a liquid extract, a solution, a suspension, or a solid form such as a simple or complex powder, a dry extract or the like, to be added to a spheroid of controlled granular size.

It is preferably used with an alcoholic or hydro-alcoholic extract of one or more primary vegetable materials. However, it is also suitable for use with any other solution or active powder containing one or more active principles, for example, vitamin, mineral and/or organic component.

One of the characteristics of the invention is to use for the dry technological mixture the specific physical-chemical properties of a slightly substituted hydroxpropylene cellulose ether at the level of the core groups β-O-glucopyranosil. This compound is preferably characterized by a substitution rate (hydroxypropyl groups) of the order of 10%, for example approximately 11%, and preferably by a water soluble fractional percentage of the order of 5%, for example, 4.29%.

This compound has plastic properties, it is liquid adsorbent and absorbent, and is compatible with the extrusion and spheronization method. Moreover, the resulting spheroids have a much higher concentration of added components in comparison to those prepared with the same method using more conventional technological excipients, particularly microcrystalline cellulose.

Below are some examples supporting this statement, which test the quantity of water absorbed by the same mass (100 g) of excipients of different types:

| | AMOUNT OF WATER ABSORBED |
|---|---|
| TYPE OF EXCIPIENT | PER 100 g OF EXCIPIENT |
| Lactose | 15 g |
| Starch | 60 g |
| Microcrystalline cellulose | 120 g |
| Hydroxypropylated cellulose | 350 g |
| ether slightly substituted (LHPC) | |

The dry mass of hydroxypropylated cellulosic polymer is wetted with a wetting liquid which may be the active solution or another aqueous or non-aqueous liquid in the case of an active powder, until a homogeneous, malleable paste is obtained which can undergo the next stages in the method, that is, extrusion and spheronization.

The wetting liquid serves as a vehicle for transporting and depositing the active substances on the core of the absorbent and adsorbent substance, in the microcavities of the hydroxypropylated celluosic polymer.

Next, the fabrication method consists of extruding the wet mass through a die with calibrated openings and then forming the extruded product into spheres (spheronization).

During the extrusion process, the wetted mass is compressed and then stretched to transform it into compact filaments of generally cylindrical cross-section known as “extrudates.”

To obtain spheroids, the “extrudates” are placed in a cylindrical apparatus called a “spheronizer” which has in its lower portion a disc rotating at a variable, controlled speed. Due to the effect of centrifugal force exerted by the rotation of the disc, the “extrudates” become regularly fragmented and then transform into spheres due to a rolling-binding effect.

It has been observed that the ability to transform the extrudates into spheroids of homogeneous sphericity and predefined, regular granular size depends as much upon the plastic qualities of the extrudates and thus, the plastic qualities of the wetted mass, as on the characteristics related to the spheronization operation per se, that is, the rotation speed of the rotating disk and the duration of rotation.

According to a feature of the invention, to obtain an adapted plasticity that is compatible with the extrusion and then spheronization techniques, the mass of the wetting liquid preferably ranges from 3 to 5 times the mass of hydroxypropylated cellulosic polymer used.

A specific feature of the invention cited by way of example consists of preparing spheroids ranging in granular size from 350 microns to 1250 microns, resulting in a yield of at least 95% of the mass of spheroids produced.

In this case the extruding die comprises orifices with openings of 1000 microns in diameter and 1000 microns long (thickness of the die). The extrusion speed is 100 rpm for a frontal dual screw or other type of extrusion apparatus.

The spheronization cycle for a spheronization apparatus with a 25 cm or other diameter is thus 5 minutes long for a rotation speed of 1040 rpm.

The spheroids are then dried at a temperature of approximately 30-40° C.

Next they are passed through a calibration device consisting, for example, of a sieve, in order to obtain multiparticular spherical systems of controlled granular size.

The resulting spheroids are stable over time, easy to control, and reproducible. They are practical and easy to use. They can be stored without any special precautions or care, and they are easy to transport because they are light, compact, and not fragile.

The spheroids according to the present invention may be presented loose or packaged in bulk, in dosage devices, capsules, tablets, packets, or any other suitable physical form or package.

They are preferably grouped in capsules or gelatin caps containing the quantity of active substances determined on the basis of the usual dosage of the compound so that a single dose is simple to take.

Before being packaged, for example, in capsule form, these multiparticular systems can be covered with film during a supplemental step in the method according to the invention.

This procedure, used primarily when the spheroids are designed for oral ingestion, consists of coating them with a film made of resistant material to protect the molecules of the active principle, which may be sensitive to the acid pH of the stomach or degraded by intestinal enzymes.

This step also regulates dispersion of the active components throughout the organism. Dispersion may be slowed in order to deliver the active principles selectively to certain levels of the digestive system, for example, the upper and median portions of the small intestine.

By using different types of coatings for spheroids within the same capsule, it is also possible to deliver several successive doses of the active principles when only one dose of the compound is administered.

According to an essential feature of the invention, the dry technological mixture comprises an ether of slightly substituted hydroxypropylated cellulose at the level of the core groups β-O-glucopyranosil, preferably characterized by a substitution rate of the order of 10%, for example approximately 11%, and a water soluble fractional percentage of approximately 5%, for example, 4.29%.

In addition to its plastic properties, liquid absorbency and adsorbency, and compatibility with the extrusion-spheronization process of the invention, this compound possesses the particularly advantageous property of expanding in water, thus acting as a catalyst for the dispersion of active absorbed and/or adsorbed substances.

Expansion actually facilitates entry of the liquid into the microcavities of the absorbent and adsorbent material and the lixiviation process of extracting the active products it carries.

This compound also permits rapid delitescent dispersion of the adsorbed and/or absorbed components in an aqueous solution.

This important feature makes it is possible to attain another goal of the invention, namely, spheroids which disperse rapidly in a liquid environment, particularly water, with or without the use of elevated temperature. The resulting liquid may be used either internally or externally to resolve problems such as those previously cited in the introduction.

For example, following oral absorption of the product, the substances are rapidly dispersed within the stomach, using digestive juices to achieve the necessary lixiviation.

All of the active principles are freed very rapidly, in a controlled and reproducible manner. This exceptional bio-availability makes the formulation original and highly effective for preventive and curative medicine.

The method of fabricating rapid dispersion spheroids according to the invention comprises the following steps:

providing an active solution or powder;

providing a dry homogeneous mixture of slightly substituted hydroxypropylated cellulosic polymer with absorbent and adsorbent properties that is compatible with the extrusion and spheronization method and allows rapid delitescent dispersion of the added components;

wetting the dry mixture with the active solution or with another aqueous or non-aqueous liquid;

extruding;

forming spheroids by spheronizing the extruded product;

drying;

calibrating the dried composition to form multiparticular spheroid systems of controlled granular size.

Here again the method may comprise a supplementary step consisting of coating the multiparticular systems with a film, particularly a film which is resistant to digestion in one or more portions of the digestive tract.

To further improve the advantageous rapid dispersion and to confer new properties to the spheroids, another feature of the invention consists of utilizing the specific physical-chemical and pharmaco-technical properties of excipients that are compatible with the spheroid formation process, as well as their ability to disperse in a liquid environment, particularly water, by rapidly disaggregating once in contact with a liquid, a complex solution, or any liquid, paste, or semi-paste compound with a high water content.

According to a preferred embodiment of the invention, the cellulosic hydroxypropylated ether is associated with at least one pharmaceutical excipient conventionally used for its high performance aqueous solubility, for example, lactose or other types of derivatives, preferably in proportions ranging generally from 20 to 50% of the total dry mass.

It can also be associated with a disintegrating excipient with “flash” delitescent properties such as reticulated sodium carboxymethylcelluose, preferably in proportions of about 5% of the total dry mass.

In order to confer additional qualities, the compound may further comprise other excipients in current use, such as binding agents, gliding agents, lubricating or surfactants, or the like.

The following examples will aid in understanding the invention.

Spheroids with a base of primary material of vegetable origin (liquid extracts), with a variable alcohol titrant and dry residue, have been prepared according to the method of the invention and with the excipient described above:

### EXAMPLE 1

| | | |
|---|---|---|
| liquid valerian extract | 350 | ml |
| alcohol titrant: 27%, dry residue: 9.1% | | |
| slightly substituted hydroxypropylated cellulosic ether | 100 | g |

### EXAMPLE 2

| | | |
|---|---|---|
| liquid red vine extract | 350 | ml |
| alcohol titrant: 11%, dry residue: 9.5% | | |
| slightly substituted hydroxypropylated cellulosic ether | 100 | g |

### EXAMPLE 3

| | | |
|---|---|---|
| liquid dandelion extract | 300 | ml |
| alcohol titrant: 40%, dry residue: 21.0% | | |
| slightly substituted hydroxypropylated cellulosic ether | 100 | g |

1. Spheroids characterized in that they are formulated from at least one active substance absorbed and/or adsorbed on a dry homogeneous technological mixture of slightly substituted cellulosic hydroxypropylated polymer and in that they are obtained using an extrusion and spheronizing method.

2. Spheroids according to the preceding claim characterized in that the active substance is of vegetable origin.

3. Spheroids according to the preceding claim characterized in that the active substance initially originates from an alcoholic or hydro-alcoholic extract of one or more primary vegetable materials.

4. Spheroids according to any one of the preceding claims characterized in that the technological mixture comprises a slightly substituted cellulosic hydroxypropylated polymer ether at the level of the core groups β-O-glucopyranosil.

5. Spheroids according to the preceding claim characterized in that the hydroxypropylated cellulosic ether has a substitution rate (hydroxypropyl groups) of the order of 10%, specifically about 1 1%.

6. Spheroids according to claim 4 or 5 characterized in that the hydroxypropylated cellulosic ether has a water soluble fractional percentage of the order of 5%, specifically, approximately 4.29%.

7. Spheroids according to any one of claims 4 through 6 characterized in that the hydroxypropylated cellulosic ether is associated with at least one pharmaceutical excipient that is conventionally used for its high performance aqueous solubility, especially with lactose.

8. Spheroids according to the preceding claim characterized in that this at least one excipient generally represents from 20 to 50% of the total dry mass.

9. Spheroids according to any one of claims 4 through 8 characterized in that the hydroxypropylated cellulosic ether is associated with a disintegrating excipient possessing “flash” delitescent properties, particularly with reticulated sodium carboxymethylcellulose.

10. Spheroids according to any one of the preceding claims characterized in that they are grouped in capsules or gelatin capsules.

11. A vegetable, mineral, vitamin, or active organic component-based compound characterized in that it takes the form of galenic spheroids according to any one of the preceding claims.

12. A method of fabricating spheroids heavily titrated with active principles, characterized in that it comprises the following steps:
providing a highly concentrated active solution or powder;
providing a dry homogeneous mixture of slightly substituted hydroxypropylated celluosic polymer with very elevated absorbent and adsorbent properties;
wetting the dry mixture with the active solution or with another aqueous or non-aqueous liquid;
extrusion;
formation of spheroids by spheronizing the extruded product;
drying
calibrating the dried compound in order to form multiparticular spherical systems of controlled granular size.

13. A method of fabricating rapid dispersion spheroids characterized in that it comprises the following steps:
providing an active solution or powder;
providing a dry homogeneous mixture of slightly substituted hydroxypropylated celluosic polymer with absorbent and adsorbent properties compatible with the extrusion and spheronization procedure and allowing rapid delitescent dispersion of the added components;
wetting the dry mixture with the active solution or with another aqueous or non-aqueous liquid;
extrusion;
formation of spheroids by spheronizing the extruded product;
drying
calibrating the dried compound in order to form multiparticular spherical systems of controlled granular size.

14. A method of fabricating spheroids according to claim 12 or 13 characterized in that it further comprises the following step:
coating the multiparticular systems with film.

15. A method of fabricating spheroids according to any one of claims 12 through 14 characterized in that the mass of the wetting liquid ranges from 3 to 5 times the mass of hydroxypropylated cellulosic polymer used.

16. A method of fabricating spheroids according to any one of claims 12 through 15 characterized in that the extrusion die comprises orifices with a 1000 micron diameter opening and 1000 microns long and in that the speed of extrusion is 100 rpm.

17. A method of fabricating spheroids according to any one of claims 12 through 17 characterized in that the spheronisation cycle effected with a 25 cm diameter spheronization apparatus is 5 minutes long at a rotation speed of 1040 rpm.

18. A method of fabricating spheroids according to any one of claims 12 through 17 characterized in that the spheroids are dried at a temperature of approximately 30-40° C.

19. A method of fabricating spheroids according to any one of claims 12 through 18 characterized in that the dried compound is calibrated to yield spheroids ranging in granular size from 350 microns to 1250 microns, for a yield of at least 95% of the mass of spheroids produced.

20. (NEW) A plurality of spheroids
wherein the spheroids are formulated from at least one active substance absorbed and/or adsorbed on a dry homogeneous technological mixture of slightly substituted cellulosic hydroxypropylated polymer and in that the spheroids are obtained using an extrusion and spheronizing method.

21. (NEW) The spheroids according to claim 20, wherein the active substance is of vegetable origin.

22. (NEW) The spheroids according to claim 20, wherein the active substance initially originates from an alcoholic or hydro-alcoholic extract of one or more primary vegetable materials.

23. (NEW) The spheroids according to claim 20, wherein the technological mixture comprises a slightly substituted cellulosic hydroxypropylated polymer ether at the level of the core groups β-O-glucopyranosil.

24. (NEW) The spheroids according to claim 20, wherein the hydroxypropylated cellulosic ether has a substitution rate (hydroxypropyl groups) of the order of 10%, specifically about 11%.

25. (NEW) The spheroids according to claim 23, wherein the hydroxypropylated cellulosic ether has a water soluble fractional percentage of the order of 5%, specifically, approximately 4.29%.

26. (NEW) The spheroids according to claims 23, wherein the hydroxypropylated cellulosic ether is associated with at least one pharmaceutical excipient that is conventionally used for its high performance aqueous solubility, especially with lactose.

27. (NEW) The spheroids according to claim 20, wherein at least one excipient generally represents from 20 to 50% of the total dry mass.

28. (NEW) The spheroids according to claim 23, wherein the hydroxypropylated cellulosic ether is associated with a disintegrating excipient possessing “flash” delitescent properties, particularly with reticulated sodium carboxymethylcellulose.

29. (NEW) The spheroids according to claim 20, wherein the spheroids are grouped in capsules or gelatin capsules.

30. (NEW) A vegetable, mineral, vitamin, or active organic component-based compound wherein the organic component-based compound is of the form of galenic spheroids, and the galenic spheroids are formulated according to the spheroids of claim 20.

31. (NEW) A method of fabricating spheroids heavily titrated with active principles, wherein the method comprises the following steps:
providing a highly concentrated active solution or powder;
providing a dry homogeneous mixture of slightly substituted hydroxypropylated celluosic polymer with very elevated absorbent and adsorbent properties;
wetting the dry mixture with the active solution or with another aqueous or non-aqueous liquid;
extrusion;
formation of spheroids by spheronizing the extruded product;
drying
calibrating the dried compound in order to form multiparticular spherical systems of controlled granular size.

32. (NEW) A method of fabricating rapid dispersion spheroids characterized in that it comprises the following steps:
providing an active solution or powder;
providing a dry homogeneous mixture of slightly substituted hydroxypropylated celluosic polymer with absorbent and adsorbent properties compatible with the extrusion and spheronization procedure and allowing rapid delitescent dispersion of the added components;
wetting the dry mixture with the active solution or with another aqueous or non-aqueous liquid;
extrusion;
formation of spheroids by spheronizing the extruded product;
drying
calibrating the dried compound in order to form multiparticular spherical systems of controlled granular size.

33. (NEW) The method of fabricating spheroids according to claim 31, wherein it further comprises the following step:
coating the multiparticular systems with film.

34. (NEW) The method of fabricating spheroids according to claim 31, wherein the mass of the wetting liquid ranges from 3 to 5 times the mass of hydroxypropylated cellulosic polymer used.

35. (NEW) The method of fabricating spheroids according to claim 31, wherein the extrusion die comprises orifices with a 1000 micron diameter opening and 1000 microns long and in that the speed of extrusion is 100 rpm.

36. (NEW) The method of fabricating spheroids according to claim 31, wherein the spheronisation cycle effected with a 25 cm diameter spheronization apparatus is 5 minutes long at a rotation speed of 1040 rpm.

37. (NEW) The method of fabricating spheroids according to claim 31, wherein the spheroids are dried at a temperature of approximately 30-40° C.

38. (NEW) The method of fabricating spheroids according to claim 31, wherein the dried compound is calibrated to yield spheroids ranging in granular size from 350 microns to 1250 microns, for a yield of at least 95% of the mass of spheroids produced.
